(19) United States
(12) Patent Application Publication
(10) Pub. No.: US 2007/0224588 A1
(43) Pub. Date: Sep. 27, 2007

(54) TROPHOBLAST PRESERVATION/PRETREATMENT MEDIUM AND METHOD

(75) Inventor: Tony Pircher, San Diego, CA (US)

Correspondence Address: COOLEY GODWARD KRONISH LLP;ATTN: Patent Group, Suite 500, 1200 - 19th Street, NW, WASHINGTON, DC, 20036-2402, US

(73) Assignee: BIOCEPT, INC., San Diego, CA (US)

(21) Appl. No.: 11/277,218

(22) Filed: Mar. 22, 2006

(51) Int. Cl. A01N 1/02; C12M 1/34; C12N 5/08

(52) U.S. Cl. 435/2; 435/366; 435/287.1

(57) ABSTRACT

An aqueous preservation medium for the selective preservation of trophoblasts obtained in a sample of cervical mucus which permits transportation of such sample to a laboratory facility for analysis and selectively preserves fetal trophoblasts in said sample while presenting conditions that are antagonistic to many maternal cells.

This invention relates to a preservation medium for preserving trophoblast cells in a sample obtained from a pregnant female mammal, and more particularly to methods for obtaining, preserving and pretreating a sample of fetal and maternal cells obtained from the uterine cavity.

### BACKGROUND OF THE INVENTION

As early as the 1970's it was known that fetal cells are shed into the endocervical canal and that it was feasible, at 8-13 weeks of gestation, to retrieve such cells for analysis to facilitate prenatal sex determination. Early efforts at such retrieval often employed flushing with sterile saline. In 1995, J. Kingdom, et al. in Obstetrics and Gynecology, 86; 2, 283-288 (1995) reported that the collection of transcervical cell samples using either endocervical saline lavage or cytobrush; it was reported that several samples collected by cytobrush contained more endocervical cells than samples collected by lavage. A standard cytology brush was inserted into the endocervical canal and rotated while being withdrawn to trap endocervical mucus. The end of the brush was cut off and placed in a small container holding about 10 ml of sterile saline. After shaking the container several times to dislodge the cells into the saline, the brush tip was removed, prior to transport of the capped container to a laboratory for analysis. Syncytiotrophoblastic cells along with others were recognized by conventional staining, and the use of polymerase chain reaction (PCR) or fluorescent in situ hybridization (FISH) was said to allow fairly accurately determination of the sex of the fetus. A companion paper by C. Rodeck, et al., Prenatal Diagnosis, 15:933-942 (1995) compared the retrieval of transcervical cells from pregnant women by lavage, mucus aspiration and cytobrush, employing both intrauterine lavage and endocervical lavage. It appeared that the use of the cytobrush was comparable to lavage for obtaining cytotrophoblasts, but was not as effective in obtaining syncytiotrophoblasts.

A group from Israel, M. D. Fejgin, et al., Prenatal Diagnosis, 2001; 21:619-621 employed the non-invasive technique of uterine cervical brushing to obtain fetal cells that were then evaluated by FISH. The cells captured by the brush were spread onto four microscope slides. It was stated that FISH analysis for common chromosomal aberrations should be a reasonable alternative to conventional karyotyping. The brushing technique was stated to be the same as that widely used to obtain a Pap smear. It was reported that the results obtained by FISH were effective in determining the sex of the fetus as well as common numerical aberrations in certain chromosomes of male fetuses. More recently a group in Italy, R. Cioni, et al., Prenatal Diagnosis (2003); 23:168-171, collected mucus samples using a cytobrush; they then treated the samples with one of two mucolytic solutions or left them without any treatment. The authors evaluated only sex determination, and they concluded that mucus sampling in this manner would yield fetal cells in such a limited number that it should not be regarded as a tool of true clinical value.

In the mid-1980's, it was proposed in (U.S. Pat. No. 4,675,286) to remove cells from the uterine cavity and the outer surface of the amniotic sac and then incubate the removed cells with antibodies that bind specifically to an antigen carried by the fetal cells, such as anti-Trop-1 and anti-Trop-2 monoclonal antibodies. In Example 5, it was proposed to mix the cells with a fluorescent-labeled antibody, incubate for 12 hours and then separate them by cell-sorting. In Example 6, the removed cells in a suitable isotonic saline were mixed with magnetic beads which carried the specific antibodies to the fetal cells and incubated for eight hours. After the magnetic beads had become coated with the fetal cells, they were separated, then suspended in a nutrient media and incubated for two weeks (see Example 7). Examination for chromosomal abnormalities was then carried out. This lengthy procedure did not achieve a high purification and was considered to be quite timestaking; as a result, faster and more accurate procedures were sought.

PCT application WO 2004/087863 proposes to diagnose for gender and potential chromosomal abnormalities by obtaining transcervical cells from a pregnant female, as by using a Pap smear cytobrush and shaking the brush into a test tube containing a few milliliters of a tissue culture medium that contains a penicillin/streptomycin antibiotic. The sample is then subjected to cytocentrifugation, and the resultant cytospin slides are kept in 95% alcohol until subjected to immunological staining, using an antibody directed against a trophoblast antigen, with numerous such antibodies being described. This staining is then followed by counterstaining the cells, as by dipping the slides in an appropriate solution so that the trophoblast cells are marked. Thereafter, in situ chromosomal and/or DNA analysis is performed upon the marked transcervical cells. Once the desired cells are marked, the staining may be removed, and FISH analysis is carried out using a two color technique and directly-labeled probes. FISH signals from such cells can be viewed using a fluorescent microscope. This course of action analyzes fetal trophoblasts essentially individually, while they remain a part of a plated mixture of fetal and maternal cells. The overall process requires much sophisticated equipment and highly trained operators, and for such reason, it is not favored.

Very generally, more fetal cells can be readily obtained from the region of the cervix than from a peripheral maternal blood sample; however, the difficulty expressed in the 2003 article, i.e. that mucus sampling yields fetal cells in too limited a number to serve as the basis for truly accurate clinical testing, still remains. As a result, improvements have continued to be sought.

### SUMMARY OF THE INVENTION

The invention provides methods for obtaining and selectively preserving fetal cells, particularly fetal trophoblasts, by obtaining a mucus sample from the cervix in a manner which has the likelihood of obtaining a fairly high percentage of trophoblasts, and then selectively preserving these trophoblasts through the use of a particular aqueous medium which is conducive to the health of trophoblasts and deleterious or antagonistic to the health of many maternal cells. Samples of cervical mucus or the like are routinely obtained in clinics, usually by use of a cytobrush, a cytobroom or a swab, and generally the ultimate testing is performed in separate laboratories. When there is interest in ultimately obtaining trophoblasts for analysis, it has been found that the use of a cytobrush can provide a superior sample. Regardless of the implement used to obtain the sample, the mixed cell population sample which is obtained at the clinic should be promptly placed in this aqueous medium, to limit exposure to the atmosphere, and transported to the laboratory at an early date so that analysis, or preparation for analysis, can be promptly undertaken. During the time of transport, when the sample is maintained within a particular temperature range, selective preservation of the desirable fetal trophoblast cells occurs, whereas the population of living maternal cells decreases so some initial selection for fetal trophoblasts occurs.

In one particular aspect, the invention provides a preservation medium for the selective preservation of trophoblasts in a sample of cervical mucus, which medium comprises: (a) a serum-free basal medium which includes an aqueous buffering system and ingredients including essential minerals, amino acids, vitamins and lipids as useful to support and grow human keratinocytes, (b) human epidermal growth factor (hEGF), (c) insulin, (d) an anticoagulant, (e) an anti-oxidant, (f) at least one antibiotic, and (g) at least one antimycotic, said preservation medium having a pH between about 7.0 and about 7.4, containing a calcium concentration not greater than about 0.2 mM, and having a dissolved oxygen content not more than about 10% of the normal dissolved oxygen content, which preservation medium is detrimental to bacteria, fungi and yeast and is not conducive to preservation of fibroblasts, red and white blood cells and various other non-trophoblast cells.

In another particular aspect, the invention provides a method for providing a sample of cells containing fetal trophoblasts from a pregnant female mammal and providing such trophoblasts separate from many other cells in said initial sample suitable for chromosomal analysis, which method comprises: taking a samples of cervical mucus on a collection device from a female subject, within about 60 seconds following obtaining a sample on said collection device, adding said sample and said collection device to a closable vessel containing a preservation medium adapted to preserve and selectively maintain fetal trophoblasts in preference to maternal cells, said medium comprising: (a) a serum-free basal medium which includes an aqueous buffering system and ingredients including essential minerals, amino acids, vitamins and lipids as useful to support and grow human keratinocytes, (b) human epidermal growth factor (hEGF), (c) insulin, (d) an anticoagulant, (e) an anti-oxidant, (f) at least one antibiotic, and (g) at least one antimycotic, said preservation medium having a pH between about 7.0 and about 7.4 containing a calcium concentration not greater than about 0.2 mM, and having a dissolved oxygen content not more than about 10% of the normal dissolved oxygen content, promptly closing said vessel to the atmosphere, maintaining said admixture of said sample and said medium at temperature between about 2° C. and about 8° C. for a period not longer than about 72 hours, during which period said trophoblasts in said sample are preserved while bacteria, fungi and yeast and many maternal cells are deleteriously affected, and separating said trophoblast cells from the collection device, the mucus and non-trophoblast cells to provide a sample wherein said trophoblasts cells can be readily subjected to chromosomal analysis.

In a further particular aspect, the invention provides a method for providing a transportation vehicle adapted to preserve and selectively maintain fetal trophoblasts in a sample of cervical mucus obtained from a pregnant female mammal at a temperature between about 2° C. and about 8° C. during transportation to a laboratory facility while bacteria, fungi, yeast and many maternal cells are deleteriously affected, which method comprises: preparing a preservation medium by admixing (a) a serum-free basal medium which includes an aqueous buffering system and ingredients including essential minerals, amino acids, vitamins and lipids as useful to support and grow human keratinocytes, (b) human epidermal growth factor (hEGF), (c) insulin, (d) an anticoagulant, (e) an anti-oxidant, (f) at least one antibiotic, and (g) at least one antimycotic, said admixture containing a calcium concentration not greater than about 0.1 mM, optionally adjusting the pH of said preservation medium so its pH is between about 7.0 and about 7.4, degassing said admixed preservation medium to have a dissolved oxygen content not more than about 10% of the normal dissolved oxygen content, filling a transportation vessel of a size sufficient to receive a device for collecting a cervical mucus sample from a pregnant human female so as to fill between about 80% and 90% of the volume thereof, closing said vessel to the atmosphere, and transporting said vehicle to a clinical facility where such mucus samples are collected.

### DETAILED DESCRIPTION OF THE PREFERRED EMBODIMENTS

Techniques for chromosomal examination of fetal cells are well established, and they can be effectively employed to determine sex and/or potential chromosomal abnormalities once pure fetal cell material is provided. Such genetic analyses that may be used include FISH, karyotyping, and molecular diagnostics such as PCR, QF-PCR and reverse transcriptase PCR.

Fetal trophoblasts have recently been targeted as prime candidates for such genetic analysis. Trophoblasts are epithelial cells that are derived from the placenta which surrounds a mammalian fetus. Trophoblast cells can generally be obtained from the uterine/cervical wall, and they may be present in a higher amount in mucus which can be found in the cervix. Three types of trophoblast cells are present in placental tissue: the villous cytotrophoblast, the syncytiotrophoblast, and the extravillous trophoblast. Villous cytotrophoblasts are specialized placental epithelial cells that differentiate, proliferate and invade the uterine wall to form the villi. Cytotrophoblasts are present in anchoring villi and can fuse to form a syncytiotrophoblast layer or columns of extravillous trophoblasts. “Trophoblast” is used to include all of these more specific types of cells.

Primary interest presently lies in transcervical cell samples which can be obtained from the cervix of a pregnant woman at an appropriate stage of gestation and which will contain viable fetal trophoblasts; however, such are relatively delicate cells and should be so treated. Such a transcervical cell sample can be obtained using one of well known cell collection techniques, e.g. by cytobrush, by cytobroom, by cytoswab and by endocervical lavage; however, the preferred method for present purposes has been found to be by cytobrushing. Cytobrushing using the usual cytobrush has been found to provide a higher percentage of fetal trophoblasts. For example, a Pap smear cytobrush may be inserted through the external os with gentle pressure to about 2 cm or until only the bottom bristles are showing, and then slowly removed while rotating it at least a full turn in one direction with gentle pressure. The conical tip end of the brush will enter the endocervical canal. Upon removal, the bristle portion of the brush, that will carry the transcervical cells caught during the collection procedure, is promptly cut off; however, for purposes of subsequent processing, it may be preferable for a small portion of the handle to be left attached to the collection portion of the brush, e.g. 1 to 3 cm of the handle. The bristle end of the brush is then deposited in a vessel or tube containing about 10 to 50 ml of Applicant's trophoblast preservation medium, and the tube is then capped, preferably within about 30 to 60 seconds following withdrawal from the cervix. When a 15 ml tube is used, it will be filled with about 13 ml of liquid, and when the collecting portion of the brush (e.g. bristles) is removed from the handle and placed in a tube, the liquid level may rise about 1 ml. The tube containing Applicant's selective trophoblast preservation medium and the brush is then promptly capped with a suitable stopper. Although such media tubes may be of any size and volume, it has been found that standard size collection tubes in either 15 ml or 50 ml volumes adequately accommodate the collection devices. Generally, a swab or a cytobrush easily fits within a 15 ml tube, while a cytobroom, which is less desired, requires the larger 50 ml tube. The 15 ml collection tube device used in concert with a cytobrush may be preferred.

This preservation medium is formulated to selectively preserve trophoblasts, as opposed to maternal cells, and employs, as a starting point, a commercially available basal medium which is sold as a serum-free basal medium having a calcium content that is strictly limited. More specifically it is important that the calcium content of the medium should not be greater than 0.1 mM and preferably should not be greater than 0.03 mM. Some commercially available basal media are formulated to include an aqueous buffering system and essential minerals, amino acids, vitamins, organic and inorganic salts and lipids and can be employed as a base component of the preservation medium. An example of such a generally satisfactory basal medium is as follows:

Low Calcium Basal Medium

| Component | g/L |
|---|---|
| Inorganic Salts | |
| Soluble Calcium | 0.005 |
| Soluble Magnesium | 0.1 |
| Potassium Chloride | 0.1 |
| Sodium Bicarbonate | 1.0 |
| Sodium Chloride | 7.5 |
| Sodium Phosphate Dibasic (anhydrous) | 0.3 |
| Minor amount of other minerals | |
| Amino Acids | |
| L-Alanine | 0.01 |
| L-Arginine (free base) | 0.2 |
| L-Asparagine (anhydrous) | 0.02 |
| L-Aspartic Acid | 0.01 |
| L-Cystine-2HCl | 0.04-0.06 |
| L-Glutamic Acid | 0.02 |
| L-Glutamine | 0.3-0.9 |
| Glycine | 0.01 |
| L-Histidine (free base) | 0.015 |
| L-Isoleucine | 0.002 |
| L-Leucine | 0.06 |
| L-Lysine HCl | 0.02 |
| L-Methionine | 0.005 |
| L-Pnenylalanine | 0.005 |
| L-Proline | 0.03 |
| L-Serine | 0.06 |
| L-Threonine | 0.01 |
| L-Tryptophan | 0.003 |
| L-Tyrosoine 2 Na—2H₂ | 0.0035 |
| L-Valine | 0.035 |
| Vitamins | |
| D-Biotin | 0.0002 |
| Choline Chloride | 0.003 |
| Folic Acid | 0.001 |
| myo-Inositol | 0.035 |
| Niacinamide | 0.001 |
| p-Amino Benzoic Acid | 0.001 |
| D-Pantothenic Acid (hemicalcium) | 0.00025 |
| Pyridoxine-HCI | 0.001 |
| Riboflavin | 0.0002 |
| Thiamine-HCI | 0.0001 |
| Vitamin B-12 | 0.000005 |
| Other | |
| D-Glucose | 1.0-2.0 |
| Glutathione (reduced) | 0.001 |
| Phenol Red-Na | 0.0053 |
| pH at RT (with sodium bicarbonate) | 7.3 .+−. 0.3 |

Such commercially available low calcium basal media suitable for serum-free growth can be obtained from Sigma-Aldrich of St. Louis, Mo., as its MCDB media line which are formulated for specific cell types; for example, item #M6395 is formulated for long term survival of human diploid fibroblast-like cells and is suitable when used with appropriate supplements. More preferred is Sigma's product #M7503, which is Sigma's version of MCDB-153 medium, which is supplied with 28 mM HEPES. This medium is one of several that were designed for the low-protein or serum-free growth of specific cell types, where there will be included hormones, growth factors, trace elements or low levels of specific protein for a particular cell type of interest. MCDB-153 is a modification of HAM's nutrient mixture, F-12, which was designed to promote the growth of human keratinocytes and other cells. It has the following formulation:

| Component | g/L |
|---|---|
| Inorganic Salts | |
| NH₄VO₃ | 0.000000585 |
| CaCl₂•2H₂O | 0.004411 |
| CuSO₄•5H₂O | 0.00000275 |
| FeSO₄•7H₂O | 0.00139 |
| MgCl•6H₂O | 0.122 |
| MnSO₄ | 0.000000151 |
| (NH₄)₄MO₄•4H₂O | 0.00000124 |
| NiCl₂•6H₂O | 0.00000012 |
| KC1 | 0.11183 |
| Na•Acetate (anhyd) | 0.30153 |
| NaCl | 7.599 |
| NaSiO₃•9H₂O | 0.000142 |
| Na₂HPO₄ (anhyd) | 0.284088 |
| Na₂SeO₃ | 0.0000038 |
| SnCl₂•2H₂O | 0.000000113 |
| ZnSO₄•7H₂O | 0.000144 |
| Amino Acids | |
| L-Alanine | 0.00891 |
| L-Arginine•HC1 | 0.2107 |
| L-Asparagine•H₂O | 0.015 |
| L-Aspartic Acid | 0.00399 |
| L-Cysteine•HC1•H₂O | 0.04204 |
| L-Glutamic Acid | 0.01471 |
| L-Glutamine | 0.8772 |
| Glycine | 0.00751 |
| L-Histidine•HC1•H₂O | 0.01677 |
| L-Isoleucine | 0.001968 |
| L-Leucine | 0.0656 |
| L-Lysine•HC1 | 0.01827 |
| L-Methionine | 0.00448 |
| L-Phenylalanine | 0.00496 |
| L-Proline | 0.03453 |
| L-Serine | 0.06306 |
| L-Threonine | 0.01191 |
| L-Tryptophan | 0.00306 |
| L-Tyrosine•Na | 0.00341 |
| L-Valine | 0.03513 |
| Vitamins | |
| D-Biotin | 0.0000146 |
| Choline Chloride | 0.01396 |
| Folic Acid | 0.00079 |
| Myo-Inositol | 0.01802 |
| Niacinamide | 0.00003663 |
| D-Pantothenic Acid•½Ca | 0.000238 |
| Pyridoxine•HC1 | 0.00006171 |
| Riboflavin | 0.0000376 |
| Thiamine•HC1 | 0.000337 |
| Vitamin B-12 | 0.000407 |
| Other | |
| Adenine•HC1 | 0.03088 |
| D-Glucose | 1.081 |
| HEPES | 6.6 |
| Phenol Red•Na | 0.001242 |
| Putrescine•2HC1 | 0.000161 |
| Pyruvic Acid•Na | 0.055 |
| Thioctic Acid | 0.000206 |
| Thymidine | 0.000727 |
| NaHCO₃ | 1.176 |

There are many commercially available versions of MCDB-1530 and very similar serum-free media that are especially formulated for culturing human keratinocytes which may be used as a base component of Applicant's preservation medium. For example, GIBCO™ keratinocyte-SFM is another that is commercially available. Very generally, such a base component will include essential minerals in the form of the package of inorganic salts, essential amino acids, vitamins and lipids and an appropriate aqueous buffering system. Preferably L-glutamine is present in a concentration of at least about 2 mM, and more preferably at least about 5 mM (0.73 g/L).

In addition to the standard base component of such a low calcium, basal medium, the preservation medium should contain various additives which are preferred for the selective preservation of fetal trophoblasts. In this respect, the medium should contain anti-apoptotic compounds which will slow down programmed cell death in trophoblasts. These compounds may include growth factors such as prolactin, placental growth hormone/placental lactogen, hepatic growth factor, epidermal growth factor (EGF) and insulin. Preferably, at least human EGF and insulin are added. Insulin should be present in an amount of at least about 1 microgram (mcg) per ml, and is preferably present at a concentration of about 5 mcg/ml or greater, and hEGF is preferably present at a concentration of at least about 5 mg/ml. The medium should also contain an anticoagulant, such as heparin, a soluble citrate or EDTA. Heparin is preferred and serves to prevent coagulation of blood that may be contained in the sample, thereby preventing the formation of insoluble clumps that may contain trophoblasts. At least one antibiotic is present to prevent the growth of bacteria, fungus and/or yeast during transportation of the sample. For example, penicillin in a concentration of at least about 100 U/ml and/or streptomycin in a concentration of at least about 100 μg/ml, along with at least one antimycotic, e.g. amphotericin B.

It has been found that trophoblasts are sensitive to oxidative stress; during the first trimester, the oxygen level in the placenta is only around 4%. Shortly before Applicant's 15 ml tubes, for example, are filled with the preservation media for shipment to a clinical facility where samples will be obtained, it is important that the media is degassed. Sparging with helium has been found to be an effective method of degassing and is preferred. Such sparging should be carried out to an extent that the amount of oxygen that remains dissolved in the liquid medium is not more than about 10% of what would be present if the liquid media would simply be allowed to be exposed to the atmosphere for an extended period of time and thus reach equilibrium. Preferably, the tube is filled to between about 80% and about 90% of its volume with the nearly oxygen-free media. Once a vessel or tube is filled to the desired level, e.g. 13 ml in a 15 ml tube, the tube is capped while preferably flowing an inert gas such as helium or nitrogen into the open upper end so that the air space in the vessel below the stopper is substantially filled with such inert gas.

Moreover, it has been found that an albumin, such as bovine serum albumin (BSA), will serve a dual function of decreasing oxidative stress and transporting nutrients to the cell while adsorbing toxic compounds that might be present in the sample. BSA is preferably included in an amount of at least about 0.1%. Bovine pituitary extract (BPE), transferring and selenium are also preferably included. Transferring is preferably present in a concentration of at least 0.005 mg/ml, and BPE is preferably present in a concentration of at least about 30 μg/ml.

After a cytobrush, for example, is deposited in the tube containing this preservative medium, the tube is promptly closed with a stopper or other cap. It is refrigerated and then transported to a laboratory in a cold-pack which will initially keep the capped sample at between about 2° C. and 8° C. The sample should not be allowed to freeze, as such may be detrimental to these fragile trophoblasts and should be avoided. If there should be an extended transportation period, the temperature in the shipping container may reach a temperature of up to about 15° C. for a short period of time without problem, and these delicate trophoblast cells can be maintained for up to about 3 days in this medium at these temperatures. However, transport to the laboratory for further processing is preferably achieved within about 24 hours, where they should be promptly returned to an environment between about 2° C. and 8° C. until ready to be processed. To facilitate transport of such samples from the clinic where they are obtained to the analytical laboratory, insulated transportation packages are preferably provided which are designed to hold multiple such tubes, for example, ten. The packages will accommodate one or two or even more cold packs in the form of refreezable, sealed liquid containers of the type generally known as “Blue Ice”. The shipment of these filled tubes to the facility may be at ambient temperature. Upon reaching the clinical facility, the refrigerant packs are removed and frozen in a laboratory freezer or the like. When samples of cervical mucus are to be obtained, one of the packages is opened, e.g. through the use of any standard zipper or Velcro closure or the like, and the cold packs are inserted. An individual tube is withdrawn upon the taking of a sample, unstoppered to allow the collection device to be deposited in the tube, and the stopper promptly replaced, preferably all within a time of about one minute. The sample tube with the collection device is then returned to a receptacle in the package proportioned to hold the tube, and at the end of the day, one or more packages, which will now have been cooled to between about 2° and 8° C. as a result of the presence of the frozen refrigerant packs in the insulated package, are shipped by overnight express to the analytical facility.

To prepare a cervical mucus sample for testing, such as karyotyping, FISH and/or molecular diagnostics, such as PCR, QF-PCR and reverse transcriptase PCR, the sample must be suitably processed to ready it for laboratory analysis. For example, trophoblast viability should be maintained while separating the fragile trophoblast cells from other biological material. Mucus samples will contain various biological components besides trophoblast cells, such as maternal cells, lysate from lytic cells, extracellular DNA, extracellular proteins, and the like potentially complicating matters. Upon arrival at the laboratory, the trophoblast cells, as well as other biological components, are at least partially embedded in cervical mucus which is composed of protein-polysaccharide complexes called mucopolysaccharides (also glycosaminoglycan) and other macromolecules.

Therefore, pretreatment of mucus samples, as described hereinafter, should advantageously be employed to eliminate certain non-cellular components while selecting for viability of trophoblasts. The use of trophoblast-enriching media and variants thereof, as described hereinbefore, has been found to be useful in preserving and cultivating delicate trophoblast cells, and the pretreatment methods described hereinafter are designed to discriminately eliminate some non-trophoblast components while concurrently purifying fragile trophoblast cells.

The following is a description of a generalized pretreatment procedure for purifying and cultivating trophoblasts from a cervical mucus sample upon arrival at a clinical laboratory. Moreover, specific examples complete with details are set forth hereinafter.

A stoppered tube containing a cervical mucus sample, the collection device, and the transport media, upon arriving at a laboratory, is logged into the laboratory's data management system. Following data entry and requisite paperwork, the tube is forwarded to laboratory technicians for the pretreatment procedure. Laboratory technicians follow safety guidelines, including the use of a biohazard hood, when such human biological materials are being handled in the pretreatment process described hereinafter.

Initially, the volume of the sample is reduced via vacuum aspiration where the top several milliliters of the sample are removed. Following sample volume reduction, chemical agents (e.g. enzymes) including, for example, nucleases, hydrolases, mucinases, and/or proteases may be added to the sample volume. In one example, nucleases may be added to the sample to enzymatically cleave extracellular single and/or double stranded DNA. Endonucleases are preferred over exonucleases because they cleave DNA at several interior positions, whereas exonucleases only digest nucleotides from the end of a DNA strand. Thus, endonucleases may provide a more thorough digestion in comparison to exonucleases. However, under certain circumstances exonucleases may be used instead of, or in addition to, endonucleases. Restriction enzymes, which are endonucleases that cleave in specific regions, may be utilized; however, non-specific endonucleases or, nickases, are preferred. In one example, deoxyribonuclease I (DNase I), a nickase, may be added to a sample tube to digest extracellular DNA. Examples of other nickases that may be used include DNase (which degrades single stranded and double stranded DNA), Mung bean nuclease (which digests single stranded DNA and RNA), and Benzonase® (which degrades DNA and RNA in many forms to small oligonucleotides and promotes quick reduction of cell lysate viscosity, which is useful for ultracentrifugation). Eurogentec North America, Inc. of San Diego markets each of the aforementioned endonucleases. Because mucus samples derived from the cervix contain a heterogeneous collection of biological material including intact cells (both maternal and fetal), lysate from lytic cells, extracellular proteins, and extracellular DNA, nucleases are useful for degrading exogenous DNA which, if not eliminated, may negatively affect test results and/or cause difficulty in sample processing. Degradation of extraneous DNA further isolates the cells of interest, i.e. trophoblasts; for example, the use of nucleases to hydrolyze extracellular DNA has been shown to degrade mucosal secretions as reported by Duplantier et al. in US Pharmacist, 17:34-52 (1992).

In another example, one or more hydrolases, including β-galactosidase may also be added to a mucosal sample. As with other hydrolases (e.g. nucleotideases), β-galactosidase cleaves substrates with the addition of H₂O at the point of cleavage. Specifically, β-galactosidase breaks the glycosidic bond between the sugar galactose from a variety of substrates, including ganglioside GM 1, lactosylceramides, lactose, and various glycoproteins and oligosaccharides at the β position. In the case of mucosal samples, β-galactosidase has been found to be useful in hydrolyzing sugar residues from proteins. The cleaving of such residues from proteins, such as those proteins which may be associated with the cellular membrane of a trophoblast cell (e.g. receptors), may render the sample further clarified for more efficient processing.

Mucolytic agents, such as N-acetyl-L-cysteine may also be added. Mucolytic agents are useful in reducing the viscosity of the cervical mucosa; thus, aiding in the physical transfer of mucin from the cytobrush into a tube for collection and further processing. It is generally understood that N-acetyl-L-cysteine liquefies mucus by breaking down mucopolysaccharides (also glycosaminoglycan) into smaller molecular subunits. Although N-acetyl-L-cysteine may perform the desired mucolytic activity, it should be understood that any number of mucinases may be used to hydrolyze cervical mucosa. Examples of other mucinases include dithiothreitol (DTT), bromhexine hydrochloride, L-cysteine, and any of the hyaluronidases, including hyaluronate lyase, hyaluronoglucosaminidase, and hyaluronglucuronidase.

Another group of chemical agents which may be used in processing mucosal samples are proteases which hydrolyze proteins. As previously mentioned, cervical mucosal samples contain extracellular proteins. Performing proteolytic cleavage on extracellular proteins yields a cleaner sample, thus, further isolating trophoblast cells from their original heterogeneous environment. In one example, the enzyme trypsin is used to digest extracellular proteins in a sample tube. Trypsin is a serine protease that hydrolyzes peptide bonds on the carboxyl side of the amino acids arginine and lysine. As is known in the art, there are numerous proteolytic compounds that are useful for hydrolyzing proteins. Many of these compounds may be used in addition to, or in lieu of trypsin; some of these compounds include chymotrypsin, pepsin, and papain. Although nucleases, mucolytic agents, galactosidases, and proteases have been found useful in the processing of mucosal samples, it should be understood that various types of chemicals may be used instead of, or in addition to, those described above. For example, depending on the specific nuclease, mucolytic agent, galactosidase, and/or protease used, different supplemental reagents and/or buffering solutions may be employed. As is known in the art, certain enzymes may require an incubation period at an elevated temperature in order for the enzyme to activate. Additionally, some enzymes, if left unchecked, have a tendency to over-degrade certain biological components. Therefore, depending on several variables including the enzyme or enzymes used, the concentration of the enzyme or enzymes used, reaction time, and temperature parameters, digestion results may vary widely. In order to stop digestion, some enzymes may require quenching by a second reagent or a significant drop in the reaction temperature. Furthermore, not all of the enzymes described hereinabove may be necessary for processing every sample of trophoblastic cells. Or, alternatively, in some cases more than one of one or more classes of enzymes may be utilized.

Although the order of adding enzymes may differ depending on various conditions and circumstances, in one example, a nuclease may be added to the sample followed by the addition of a galactosidase. The resultant mixture may then be placed on a rocker for an amount of time to ensure an even distribution of reagents and for the enzymatic reactions to occur. The rocker is preferably set at a temperature above ambient temperature, e.g. about 37° C., to promote the enzymatic reactions. Other of the aforementioned may then be used to similarly treat the sample, either simultaneously or sequentially, as desired.

After enzymatic treatment of the sample has been completed, the collecting device (e.g. cytobrush, cytobroom, or swab) is removed from the tube and placed into a pre-labeled sterile tube. The tube (without the collection device) is then centrifuged to concentrate the biological material into a pellet at the bottom of the tube. After centrifugation and one or more washing steps, the supernatant in the tube is removed by vacuum aspiration leaving only the pellet and a minimum amount of media. The pellet and remaining media are resuspended in a solution of MEMS buffer and DNase I. The sample is triturated using a serological pipette.

The sample is again centrifuged to wash and reconcentrate the biological material into a pellet at the bottom of the tube. Following centrifugation, the supernatant is removed from the tube via vacuum aspiration leaving an intact pellet in a minimal amount of media. The pellet and remaining media are resuspended in fresh MEMS buffer.

The final volume of the trophoblast solution pretreated according to the above is subsequently run through a microchannel separation device, such as that disclosed in pending U.S. patent application Ser. No. 11/038,920, the disclosure of which is incorporated herein by reference. The microchannel is coated with antibodies (Abs) specific for trophoblast antigens for the purpose of retaining trophoblast cells in the microchannel while allowing the media to pass through the microchannel. Examples of trophoblast specific Abs that may be used include anti-Trop-1 and anti-Trop-2, as well as those listed in WO'863.

Trophoblasts separated in such a microchannel device may be fixed for FISH analysis, where FISH analysis may be performed inside the microchannel. However, other types of genetic screening, analysis, and tests may also be performed on the trophoblasts prepared in the manner set forth herein. Because the chemical agents, enriching media, and methodologies taught herein promote preservation and separation of fetal cells, the resulting purified fetal cell samples maybe used in a variety of laboratory examinations. Thus, provided herein are protocols for providing a high quality, high yielding trophoblast culture suitable for molecular analysis.

Although the invention has been described with regard to certain preferred embodiments which constitute the best mode known to the Applicant for carrying out the invention, it should be understood that various changes and modifications as would be obvious to one having ordinary skill in this art may be made without departing from the scope of the invention, which is set forth in the claims appended hereto. For example, although several commercially available media were described as constituting suitable base components upon which the formulation of the preservation media may be made, the highly specific amounts set forth in those formulae are not considered to be critical, and deviations from them by about +/−10% may be made without significantly affecting the ability of this novel solution to very effectively selectively preserve fetal trophoblasts obtained via a sample of cervical mucus. The disclosures of all patents, patent applications, and published references that appear hereinbefore are expressly incorporated herein by reference.

Particular features of the invention are emphasized in the claims which follow.

1. A preservation medium for the selective preservation of trophoblasts in a sample of cervical mucus, which medium comprises:
(a) a serum-free basal medium which includes an aqueous buffering system and ingredients including essential minerals, amino acids, vitamins and lipids as useful to support and grow human keratinocytes,
(b) human epidermal growth factor (hEGF),
(c) insulin,
(d) an anticoagulant,
(e) an anti-oxidant,
(f) at least one antibiotic, and
(g) at least one antimycotic,
said preservation medium having a pH between about 7.0 and about 7.4, containing a calcium concentration not greater than about 0.2 mM, and having a dissolved oxygen content not more than about 10% of the normal dissolved oxygen content,
which preservation medium is detrimental to bacteria, fungi and yeast and is not conducive to preservation of fibroblasts, red and white blood cells and various other non-trophoblast cells.

2. The preservation medium according to claim 1 wherein said basal medium includes, in solution, sugars and ATP and has an osmolarity between about 280 mOsm/Kg and about 310 mOsm/Kg.

3. The preservation medium according to claim 1 wherein said insulin is present in a concentration of at least about 1 mcg/ml, and said hEGF is present in a concentration of at least about 5 mg/ml.

4. The preservation medium according to claim 1 wherein said anticoagulant is heparin.

5. The preservation medium according to claim 4 wherein said antioxidant includes albumin in a concentration of at least about 1 mg/ml.

6. The preservation medium according to claim 1 wherein said at least one antibiotic includes penicillin in a concentration of at least about 100 U/ml and streptomycin in a concentration of at least about 100 μg/ml.

7. The preservation medium according to claim 1 wherein L- glutamine, one of said amino acids, is present in a concentration of at least about 2 mM.

8. The preservation medium according to claim 1 wherein said at least one antimycotic includes amphotericin B.

9. A transportation vehicle for shipping a sample of cervical mucus, which comprises:
a tubular vessel proportioned to accommodate the brush portion of a collection device and filled to between about 80% and 90% of its capacity with the preservation medium of claim 1, and
stopper means closing and seating an open end of said vessel.

10. The vehicle of claim 9 wherein air space in said vessel between the upper surface of said medium and said stopper means is substantially filled with an inert gas.

11. A package for shipping a plurality of samples of cervical mucus to a facility for analysis, which package comprises:
a plurality of the vehicles of claim 9,
an insulated shipping container for holding said plurality of vehicles, and
a refrigerant for maintaining said plurality of vehicles at a temperature between about 2° C. and about 8° C. for at least 72 hours within said insulated container.

12. A method for providing a sample of cells containing fetal trophoblasts from a pregnant female mammal and providing such trophoblasts separate from many other cells in said initial sample suitable for chromosomal analysis, which method comprises:
taking a samples of cervical mucus on a collection device from a female subject, within about 60 seconds following obtaining a sample on said collection device, adding said sample and said collection device to a closable vessel containing a preservation medium adapted to preserve and selectively maintain fetal trophoblasts in preference to maternal cells,
said medium comprising:
(a) a serum-free basal medium which includes an aqueous buffering system and ingredients including essential minerals, amino acids, vitamins and lipids as useful to support and grow human keratinocytes,
(b) human epidermal growth factor (hEGF),
(c) insulin,
(d) an anticoagulant,
(e) an anti-oxidant,
(f) at least one antibiotic, and
(g) at least one antimycotic,
said preservation medium having a pH between about 7.0 and about 7.4 containing a calcium concentration not greater than about 0.2 mM, and having a dissolved oxygen content not more than about 10% of the normal dissolved oxygen content,
promptly closing said vessel to the atmosphere,
maintaining said admixture of said sample and said medium at temperature between about 2° C. and about 8° C. for a period not longer than about 72 hours, during which period said trophoblasts in said sample are preserved while bacteria, fungi and yeast and many maternal cells are deleteriously affected, and
separating said trophoblast cells from the collection device, the mucus and non-trophoblast cells to provide a sample wherein said trophoblasts cells can be readily subjected to chromosomal analysis.

13. The method of claim 12 wherein said sample of cervical mucus is obtained on a brush having a handle portion that is removed before said vessel is closed.

14. The method according to claim 12 wherein said separating includes the steps of treating said sample in said preservation medium with a mucinase, a nuclease and/or a protease, mixing and effecting digestion for not longer than a certain time period,
then adding a stabilizing agent to the sample and mixing,
removing said collection device, and
centrifuging to provide a sample in which trophoblasts and from which bacteria, proteinaceous material, extracellular DNA and many maternal cells from the cervical mucus sample have been eliminated.

15. A method for providing a transportation vehicle adapted to preserve and selectively maintain fetal trophoblasts in a sample of cervical mucus obtained from a pregnant female mammal at a temperature between about 2° C. and about 8° C. during transportation to a laboratory facility while bacteria, fungi, yeast and many maternal cells are deleteriously affected, which method comprises:
preparing a preservation medium by admixing
(a) a serum-free basal medium which includes an aqueous buffering system and ingredients including essential minerals, amino acids, vitamins and lipids as useful to support and grow human keratinocytes,
(b) human epidermal growth factor (hEGF),
(c) insulin,
(d) an anticoagulant,
(e) an anti-oxidant,
(f) at least one antibiotic, and
(g) at least one antimycotic,
said admixture containing a calcium concentration not greater than about 0.1 mM,
optionally adjusting the pH of said preservation medium so its pH is between about 7.0 and about 7.4,
degassing said admixed preservation medium to have a dissolved oxygen content not more than about 10% of the normal dissolved oxygen content,
filling a transportation vessel of a size sufficient to receive a device for collecting a cervical mucus sample from a pregnant human female so as to fill between about 80% and 90% of the volume thereof,
closing said vessel to the atmosphere, and
transporting said vehicle to a clinical facility where such mucus samples are collected.

16. The method according to claim 15 wherein said degassing is by gas sparging with an inert gas.

17. The method according to claim 16 wherein said degassing is by helium sparging with an inert gas.

18. The method according to claim 16 wherein said vessel is closed with said medium under an inert gas atmosphere.
